(19) United States
(12) Patent Application Publication
(10) Pub. No.: US 2005/0215955 A1
(43) Pub. Date: Sep. 29, 2005

(54) Injector in "credit card" shape with shielded needle

(76) Inventor: Adam Slawson, London (GB)

Correspondence Address: HUSCH & EPPENBERGER, LLC, 190 CARONDELET PLAZA, SUITE 600, ST. LOUIS, MO, 63105-3441, US

(21) Appl. No.: 10/513,650

(22) PCT Filed: May 7, 2003
(86) PCT No.: PCT/EP03/04856

§ 371 Date: Mar. 30, 2005

(30) Foreign Application Priority Data
May 9, 2002 (GB) 0210631.8

(51) Int. Cl. A61M 5/32

(52) U.S. Cl. 604/192

(57) ABSTRACT

An injector device (10) is made in a flat credit card shape (11) with a shielded injection needle (22) which is brought out of its shielded position into an unshielded position for use.

This invention relates to injector devices. In particular this invention relates to injector devices which are made in a “credit card” shape to be convenient and relatively inconspicuous.

Injector devices for the injection of a medicament into a patient's body, for example a hypodermic syringe in combination with an injection needle, are well known. Such syringes generally comprise a barrel defining a reservoir for the medicament, with an internal piston which can be moved along inside the barrel toward the needle, i.e. in the ejection direction, to eject the medicament content through the needle. The piston is normally engaged or engageable with a plunger shaft moveable toward the syringe, to urge the piston in the ejecting direction.

It is common for users requiring administration of a medicament at an unpredictable time or in response to an emergency to carry with them an injector device loaded with a suitable medicament to inject themselves with in the event of need or an emergency. For example diabetics may need to inject themselves with insulin. A particular emergency is anaphylaxis, a severe allergic reaction that affects the whole body, usually within minutes of exposure to an allergen but sometimes after a delay. The causes of anaphylaxis include certain foods, insect stings and certain drugs, and the remedy is an injection of adrenalin as soon as possible as an emergency measure.

A problem with known injector devices is their lack of portability and their appearance, which can cause embarrassment to a user. Commonly used syringes of the usual type with a cylindrical barrel with an injection needle at one end and an internal piston driven by a plunger can also be fragile and of an awkward shape for convenient carriage by a user in for example a pocket or handbag etc.

It is an object of the present invention to provide an improved and alternative construction of injector device which addresses the problems of such known injector devices.

According to this invention an injector device is provided comprising;
a substantially planar body,
a syringe comprising a reservoir for an injectable medicament and an injection needle in communication with the reservoir such that medicament content in the reservoir can be expelled from the reservoir via the needle,
the syringe being moveable between a first configuration in which the body and the syringe are substantially coplanar and the needle has its point shielded,
and a second configuration in which the needle is exposed for use for an injection to be administered.

According to a first embodiment the injector device comprises;
a substantially planar body,
at least one arm extending in a length direction from the body in a direction substantially coplanar to the body,
a syringe comprising a reservoir for an injectable medicament and an injection needle in communication with the reservoir such that medicament content in the reservoir can be expelled from the reservoir via the needle,
the syringe being rotatably connected to the arm to be rotatable about a rotation axis which is transverse to the arm and substantially coplanar with the body so that on rotation of the syringe the needle follows an arc of rotation out of the plane of the body,
and wherein in a first configuration the body and the syringe are substantially coplanar and the needle is in a first position in which it is oriented substantially in the syringe-toward-body direction and is adjacent to the body and has its point shielded,
and the syringe is rotatable about the axis into a second configuration in which the needle is in a second position in which the needle is exposed for use.

The device of this first embodiment operates as follows. The device is provided for use with its syringe in the first configuration ready loaded with a medicament, and with its needle shielded by the body. The syringe is then rotated into its second configuration so that the needle is exposed, available for use in an un-shielded position i.e. able to be inserted into a user's skin for the injection to be administered, preferably with the syringe, body and needle coplanar and the needle pointing in the body-toward-syringe direction away from the body, typically involving a 180° rotation. The needle can then be inserted through the user's skin to administer the injection in an otherwise conventional manner.

According to a second embodiment the injector device comprises;
a substantially planar body incorporating a syringe comprising a reservoir for an injectable medicament and an injection needle in communication with the reservoir such that medicament content in the reservoir can be expelled from the reservoir via the needle,
the syringe being slideably moveable within the body between a first configuration in which the body and the syringe are substantially coplanar and the needle has its point shielded, and a second configuration in which the needle is exposed for use for an injection to be administered.

The device of this second embodiment operates as follows. The device is provided for use with its syringe in the first configuration ready loaded with a medicament, and with its needle shielded by the body. The syringe is then slideably moved into its second configuration so that the needle is exposed, available for use in an un-shielded position i.e. able to be inserted into a user's skin for the injection to be administered, preferably with the syringe, body and needle coplanar and the needle pointing away from the body. The needle can then be inserted through the user's skin to administer the injection in an otherwise conventional manner.

Preferably the body in both embodiments is substantially square or rectangular, i.e. two long sides and two short sides, optionally with rounded corners, being overall of dimensions substantially of a standard “credit card” length and width (e.g. ca. 86×54 mm), and as thin as is practical to contain a suitable volume for delivery, e.g. 1-3 mm thick. Such a shape and dimensions enable the device to be conveniently and inconspicuously kept in e.g. a handbag, wallet, purse or credit card holder or the like.

With the first embodiment preferably the overall shape of the device, i.e. the body, the one or more arm, and the syringe when in its first configuration and preferably also when in its second configuration, are together of an overall rectangular shape. Preferably in this first embodiment there are two parallel arms symmetrically disposed on opposite sides of the body. For example preferably two such arms are symmetrically oppositely disposed about the length axis of such a rectangular, e.g. “credit card” shape, i.e. such that the body and two such arms form a frame of substantially a “U” shape, with the syringe mounted for rotation between the two arms. Preferably the axis of rotation is perpendicular to the arm, preferably perpendicular to both of two arms, preferably perpendicular to the length direction of such a rectangular shape. In both embodiments the syringe may comprise a collapsible capsule from which medicament can be ejected by squeezing, e.g. a flexible sided envelope or bag from which a user can expel the medicament by squeezing the flexible side(s).

Preferably in both embodiments the syringe comprises a barrel with an internal piston which can be moved along inside the barrel toward the needle, i.e. in the ejection direction, to eject the medicament content through the needle. When the syringe is of this latter type then preferably the body incorporates a plunger shaft moveable toward the syringe, i.e. in the first embodiment in the body-toward-syringe direction e.g. slideably moveable within a longitudinal guide slot, and which at least when the syringe is in its second configuration can be moved in this direction to urge the piston in the ejecting direction.

When the device is of the overall “credit card” shape the syringe may be of a flattened generally rectangular cross section with its long sides parallel to the plane of the body, with a correspondingly sectioned barrel and piston therein.

In the first embodiment preferably the syringe is rotatably mounted on the arm, or between two arms, by means of a stub axle on the syringe or arm engaging with a corresponding socket in the arm or syringe. Such a stub axle and socket are suitably oriented transverse to the arm to define the rotation axis. Such a stub axle may be made integrally with the body and syringe.

In the second embodiment preferably the body comprises a syringe cavity within which the syringe is contained within the body and slideably moveable therein, preferably in the direction of the length axis of such a rectangular, e.g. “credit card” shape. Such a cavity may for example comprise a generally slot-form cavity elongate in this direction, and preferably provided with guide means to guide the syringe as it slideably moves and to retain the syringe within the cavity. Such a cavity may also communicate with a plunger cavity, coaxial with the syringe cavity containing the plunger shaft and within which the plunger shaft is moveable by an urging force applied by a user thereto toward the syringe to engage the piston to urge the piston in the ejecting direction.

This second embodiment may be provided with a mechanism such that the plunger is able to move the piston within the barrel in the injecting direction toward the needle, and when the plunger is subsequently moved in the opposite direction from the injecting direction the plunger and/or the piston engage with the syringe barrel to move the syringe back toward its first configuration. Such a mechanism may comprise a piston one-way mechanism, i.e. allowing movement of the piston within the barrel only in the ejecting direction. Many such mechanisms are known, e.g. one-way barbs.

The injector device may incorporate various safety features as for example as described below.

Preferably in its first configuration the syringe may be releasably locked against movement into the second configuration, e.g. rotation in the first embodiment and sliding movement in the second embodiment by a lock means which can be released prior to use. Such a lock means provides a safety means locking the needle into its sheathed position-until required for use. Various types of releasable lock means will be apparent to those skilled in the art. For example the lock means may comprise a peel-off adhesive tape between the body and the syringe holding the body and syringe together in the first configuration until the tape is removed. Such a tape may provide a convenient tamper-evidence means. For example the lock means may comprise a pin on the syringe or body which in the first configuration engages with a corresponding socket in the body or syringe to prevent movement, and which is disengaged to allow movement.

For example in the first embodiment, when the syringe is mounted by means of one or more stub axle, such a pin may engage with a socket, and be released by a relative movement of the syringe in the body-toward-syringe direction. To facilitate such movement the socket into which the stub axle fits may need to be elongated in the body-syringe direction to allow the stub axle to move longitudinally within the socket. Such a pin may be made integrally with the body and syringe.

After use the syringe may be returned, e.g. in the first embodiment rotated or in the second embodiment slid, back into its first configuration and the lock re-engaged to place the needle into a position in which it is shielded, e.g. by the body.

A further safety means may be provided in the first embodiment by a one-way rotation mechanism between the syringe and the body, so that after use the syringe may be rotated non-reversibly along the same arc into a third configuration in which the syringe is locked with its needle in a shielded position. This can provide a single use safety means. Such a one-way mechanism may be provided by a ratchet system between the stub axle and the socket.

Preferably there may be also a locking mechanism, e.g. friction or snap engaging parts, or an engaging pin and socket, by which the syringe can be releasably locked into its second configuration to facilitate performing the injection. For example such a mechanism can ensure that the syringe and body are suitably aligned and rigid during the injection.

For example in the second embodiment the device may be provided with a mechanism such that when the syringe has been moved back toward its first configuration it may be locked in this position. Additionally or alternatively for example the connection between the plunger and the piston may be severable after use to inhibit re-use. Suitable mechanisms to achieve this will be apparent to those skilled in the art, for example one-way engagement barbs on the piston.

As a safety feature in the second embodiment the syringe may be biased e.g. by a spring, an elastomeric bias, or by compression of air as the syringe is moved into its second configuration, to be returned toward its first configuration after an urging force urging the syringe toward its second configuration as been removed.

The needle and the reservoir will normally be provided sterile for use, and preferably in its first configuration the needle, or at least its point, is shielded by providing a sterile envelope around the needle, or at least around its point. Such a sterile envelope may comprise a tearable foil cover, and may also comprise an adjacent part of the body to preserve this sterility prior to use. Such a foil cover may be ruptured, e.g. torn or punctured by the needle as the syringe moves, e.g. rotates or slides toward its second configuration. Such a tearable envelope can also provide further tamper evidence.

For example in its first configuration the needle may be shielded by being enclosed within a cavity in the body which is closed by such a foil.

For example in the first embodiment, in its first configuration the needle may be received within a cavity e.g. a trough corresponding generally to the elongate shape of the needle, and the cavity may be closed by such a tearable foil cover, and the rotation of the syringe with consequent movement of the needle may force the needle through the foil cover.

For example in the second embodiment, in its first configuration the needle may be received within a cavity within the body, e.g. that in which the syringe slideably moves, the cavity may be closed by such a tearable foil cover, and the movement of the syringe in a direction along the length axis of the needle may force the needle through, i.e. puncturing, the foil cover.

To further ensure sterility the device may be provided for use sealed in a sterile pack.

By “shielded” herein is meant the needle being is in such close proximity to the body, e.g. parallel to the plane of the body and immediately adjacent to the body, or preferably received in a cavity e.g. as described above and preferably forming part of a sterile envelope, that it is in practice unlikely, preferably impossible, for the needle to accidentally puncture the skin.

For example in the above embodiments two or more of the injector devices may be provided linked together e.g. by a severable link, for example each device containing one unit of a multi-unit dose of the medicament. For example the bodies of two or more devices, may be so linked together, especially two or more generally rectangular bodies may be linked together along an edge.

For example in the above embodiments a device may incorporate two or more syringes, so that they may be used sequentially to administer sequential doses of the same or a different medicament.

The body and syringe may be made of conventional materials such as plastics materials. The syringe should be made of materials compatible with the medicament, and the needle will normally be metal. The injector device appears to be suitable for use with all types of injectable medicament, for example adrenalin for use in the treatment of anaphylaxis, or insulin for use in the treatment of diabetes.

The invention will now be described by way of example only with reference to the accompanying drawings which show:

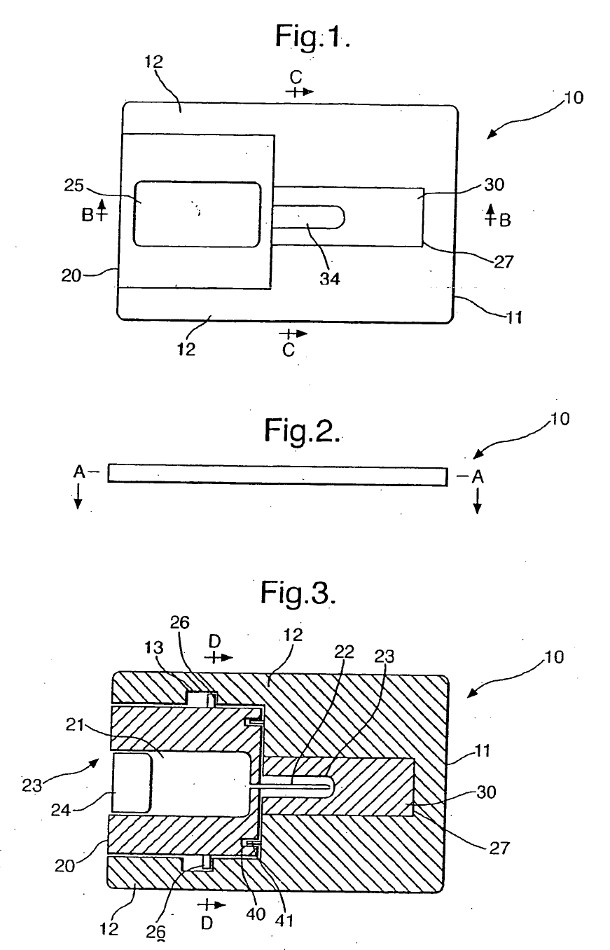

FIG. 1A plan view of a device of the first embodiment of this invention

FIG. 2A side view of the device of FIG. 1

FIG. 3A cross section of the device of FIGS. 1 and 2, along the line A-A of FIG. 2 looking in the direction of the arrows.

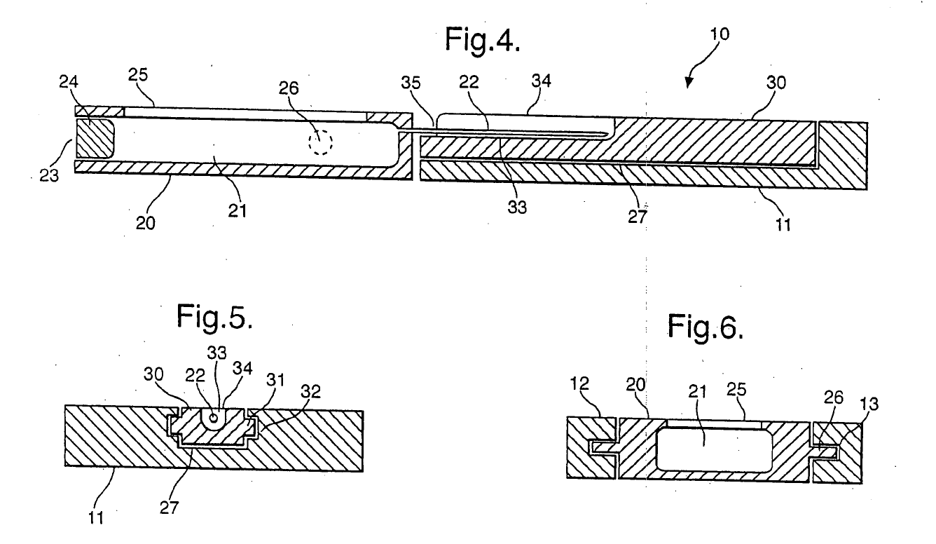

FIG. 4A cross section of the device of FIGS. 1 and 2, along the line B-B of FIG. 1 looking in the direction of the arrow.

FIG. 5A cross section of the device of FIGS. 1 and 2, along the line C-C of FIG. 1 looking in the direction of the arrow.

FIG. 6A cross section of the device of FIGS. 1 and 2, along the line D-D of FIG. 1 looking in the direction of the arrow.

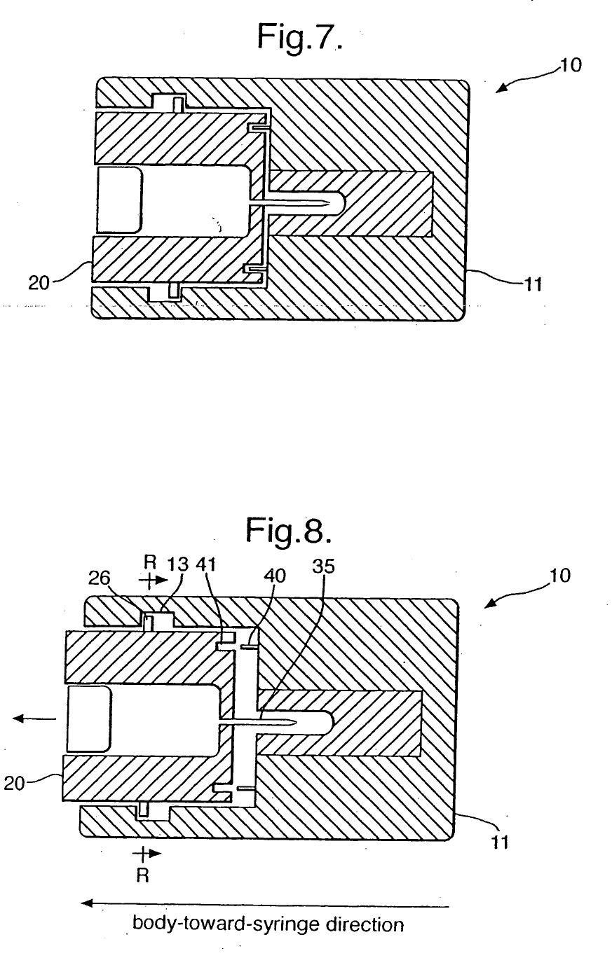

FIGS. 7 to 10 Sequential drawings showing the operation of the device of FIGS. 1-6.

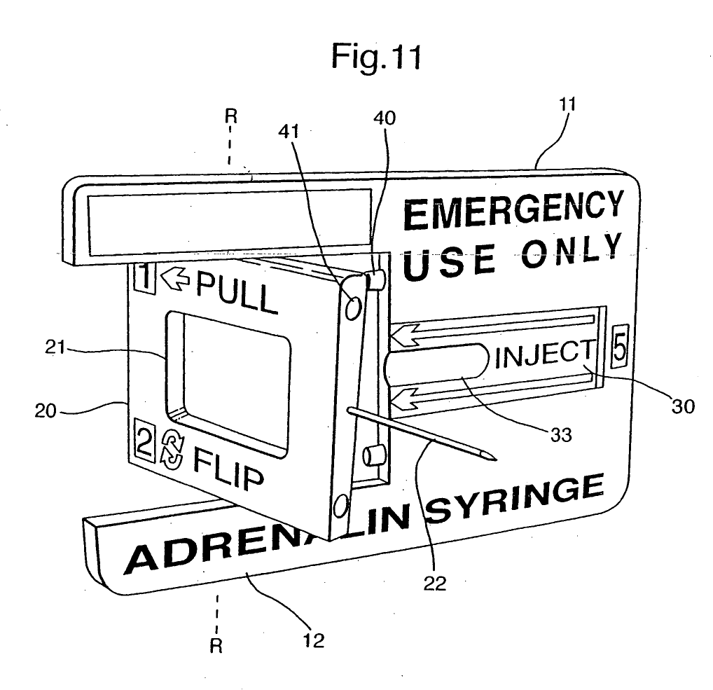

FIG. 11 shows a perspective view of the device of FIGS. 1-7.

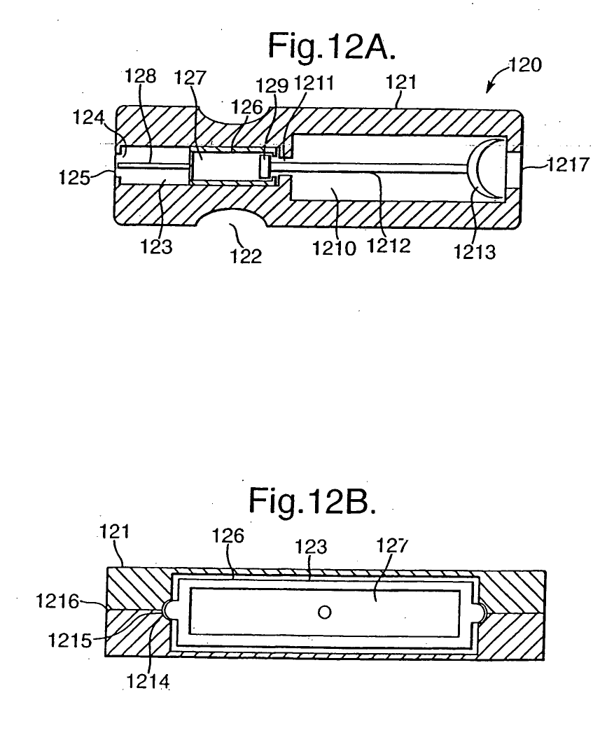

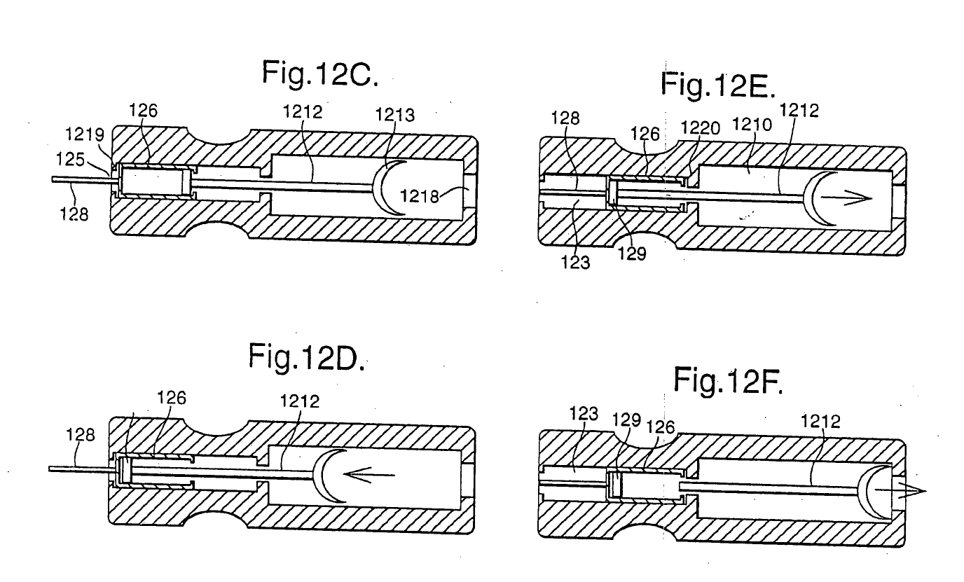

FIG. 12 shows the construction and sequential operation of a device of the second embodiment of this invention.

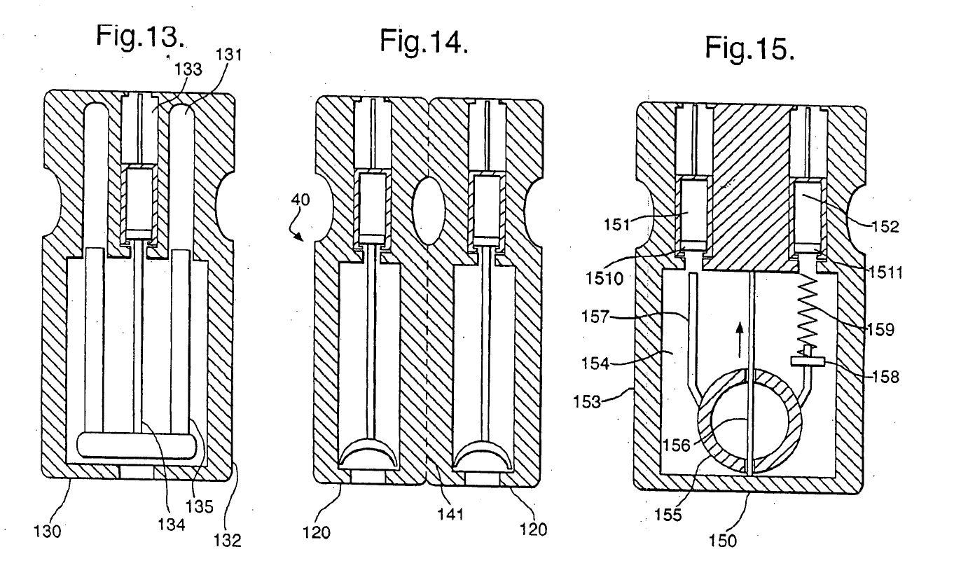

FIGS. 13-15 show further forms of a device of the second embodiment of this invention.

Referring to FIGS. 1-10 a device 10 according to the first embodiment of this invention is shown in an overall plan view in FIG. 1, and in an overall side view in FIG. 2. This shows the overall “credit card” shape and size of the device in its first configuration, i.e. a rectangle with two long sides and two short sides, and ca. 3 mm thick. FIGS. 1 to 7 show the device in a first configuration.

The device 10 comprises a substantially planar body 11 made of plastics materials, and of a generally square shape, from which extend two parallel arms 12; The arms 12 are integrally made with body 11 and are coplanar with body 11. The arms 12 are oppositely disposed about the long axis of the rectangular shape and extend in the longitudinal direction of the rectangle, such that the body 11 and the two arms 12 form a frame of substantially a “U” shape.

Mounted between the arms 12 is a syringe 20, which comprises a barrel 21 for containing an injectable medicament, which as can be seen from FIG. 6 is substantially rectangular in cross section. One end of the barrel 21 is closed but communicates with a hollow injection needle 22 which extends from the syringe 20 in the syringe-toward-body direction to its point 22A, the other end 23 of barrel 21 being open. Within the barrel 21 is a piston 24 which is slideable within the barrel 21 and which can be moved toward the closed end of the barrel 21 to expel medicament content from the barrel 21 via the needle 22. Part 25 of the wall of the barrel 21 is formed as a transparent window so that the user can observe the progress of the piston 24 toward the needle 22.

In an alternative construction (not shown) the syringe 20 may have a reservoir comprising a flexible sided bag connected to the needle.

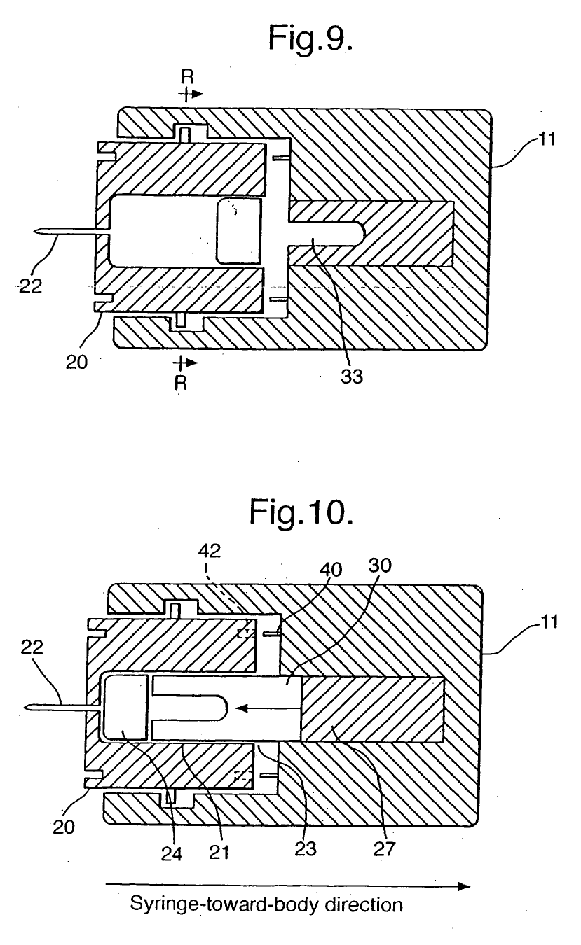

The syringe 20 is rotatably connected to the arms 12 to be rotatable about a rotation axis R-R seen in FIGS. 8 and 9 which is perpendicular to the arms 12 and substantially coplanar with the body 11. This is achieved by means of oppositely disposed stub axles 26 on the syringe 20 which engage with corresponding sockets 13 in each arm. The stub axles 26 are oriented perpendicular to the arms 12 to define the rotation axis.

Mounted in a slot 27 in body 11 is a plunger shaft 30 slideably moveable in the body-toward-syringe direction, for example having side ribs 31 which engage with corresponding grooves 32 in the sides of slot 27.

In the shaft 30 is a trough 33 which receives the needle 22, and thereby shields the needle 22. A sterile envelope is formed around the needle by the sides of the trough 33 and a tearable foil cover 34 sealed against the sides of the trough 33 and around the needle at 35. This sterile envelope further shields needle 22 and keeps the needle 22 sterile prior to use.

In the first configuration illustrated in FIGS. 1 to 7 the syringe 20 is releasably locked against rotation by a lock means. This comprises two pins 40 on the body extending in the longitudinal direction which engage with corresponding oppositely facing sockets 41 in the syringe 20 to prevent rotation. As can be seen in FIG. 8 the pins 40 and sockets 41 are disengaged by a relative movement of the syringe 20 in the body-toward-syringe direction. To facilitate such movement the sockets 13 into which the stub axles 26 fit are elongated in the body-syringe direction to allow the stub axle 26 to move longitudinally within the socket 13.

The operation of the device will now be described with reference to FIGS. 7 to 10.

As mentioned above FIG. 7 shows the device in its first configuration. As seen in FIG. 8 the syringe 20 is first moved in the body-toward-syringe direction to disengage pins 40 and sockets 41. This movement also ruptures the sterile seal at 35. To facilitate this movement the sockets 13 are longitudinally elongate so that axles 26 can move longitudinally therein.

As seen in FIG. 9 the syringe 20 is then rotated about the axis R-R through 180° into its second configuration. Needle 22 follows an arc of rotation out of the plane of the body and, tears through the foil seal 34 (providing tamper/use evidence) over trough 33, until the syringe 20 and body 11 are coplanar and the needle 22 is in the position shown, pointing in the body-toward-syringe direction away from the body and exposed ready for use and able to be inserted into a user's skin for the injection to be administered.

As shown in FIG. 10, after insertion into the user's skin (not shown) the plunger shaft 30 is then moved in the body-toward-syringe direction, so that it enters the open end 23 of the barrel 21 and on further movement in this direction drives the piston toward the needle 22 to eject the medicament therefrom. In an alternative construction the slot 27 may extend to the adjacent short side of the body 11 to allow the user to push the end of the plunger shaft remote from the piston 24.

After this use the device 10 may be disposed of, but as a safety measure the shaft 30 may be withdrawn from barrel 21 into its original position as shown in FIG. 7, and the syringe 20 may be rotated back in the opposite direction so that the needle 22 is received by the trough 33, and the syringe 20 then moved back in the syringe-toward-body direction to re-engage the pins 40 and sockets 41 to lock the device against subsequent use.

The syringe 20 may also have sockets 42 in its edge to that in which sockets 41 are situated, which can engage with pins 40 by a movement of the syringe in the syringe-toward-body direction whilst in its second configuration as shown in FIG. 10, to hold the syringe 20 in a suitable alignment with the plunger shaft 30 for the entry of shaft 30 into barrel 21.

Referring to FIG. 11 this shows the device 10 overall, with its syringe 20 in the process of being rotated from its first configuration towards its second configuration relative to body 11. Various features referred to in FIGS. 1-10 are indicated.

Referring to FIG. 12 an injector device 120 overall according to the second embodiment is shown in a plan view showing its internal construction comprises a substantially planar body 121 which is generally of a rectangular shape as seen in plan view in FIG. 12. Though shown relatively elongate the body 121 may be of a generally “credit card” shape and size. The rectangular shape is distorted by two notches 122 on opposite sides of the long axis and which assist the user in using the device 120.

The body 120 incorporates a syringe cavity 123 which is generally slot-form and elongate in the length direction of the body 120 and with longitudinal straight sides. At one longitudinal end of the cavity 123 there is an opening 124 which is closed by a tearable foil cover 125 forming a sterile seal with the adjacent rim part of the opening, so that the cavity 123 may be sterile isolated from the ambient environment.

Within syringe cavity 123 is located a syringe comprising a barrel 126 defining a reservoir 127 for an injectable medicament (not shown) and an injection needle 128 in communication with the reservoir 127 such that medicament content in the reservoir can be expelled from the reservoir 127 via the needle 128, i.e. as above. The syringe includes a moveable piston 129 which can be moved within the barrel 126 toward needle 128 in a conventional manner.

The syringe is contained within the syringe cavity 123 in body 121, and is slideably moveable therein along the direction of the length axis of the cavity 123. To facilitate this the sides, e.g. the long sides of the slot, and the barrel 126 may be provided with guides (not shown in FIG. 12A), e.g. a guide groove and a corresponding co-operating external part of the barrel.

The body 121 also includes a plunger cavity 1210, again generally elongate slot form, and in communication at 1211 with cavity 123. Cavity 1211 encloses a plunger shaft 1212 which is connected to the piston 129. The plunger shaft 1212 is longitudinally moveable within cavity 1210 by an urging force applied by a user thereto, e.g. via button 1213, toward the syringe to urge the piston 129 in the injecting direction to conventionally expel medicament through needle 128.

FIG. 12B shows an enlarged cross section of the device 120 at line A-A showing the overall rectangular section of the body 120, guide grooves 1214 in the sides of cavity 123 and corresponding convexities 1215 in the sides of the barrel 126. FIG. 12A also shows how the body 120 may be in two-part construction joined at 1216 e.g. by welds.

As seen in FIG. 12A the syringe (126, 127, 128, 129) is in a first configuration in which the needle 128 has its point shielded by being enclosed within the cavity 123, the opening 124 being closed by foil cover 125.

A sterile seal cover e.g. a peel-off film may be provided over the cavity 1210 to protect the sterility of the needle 128. Alternatively or additionally a sterile sliding seal e.g. an elastomer washer may be provided between the barrel 126 and the side of the cavity 123, and/or at the communication 1211.

The operation of the device of FIG. 12 will now be described.

As shown in FIG. 12C, A tamper evidence cover, e.g. a snap-off cover 1217 has been removed to allow access via opening 1218 to button 1213. The syringe (126, 127, 128, 129) is then moved longitudinally by urging the plunger 1212 via application of force to button 1213, so that the needle 128 punctures the foil cover 124 and is exposed for use. At the extreme end of this movement the barrel abuts against end-stops 1219 at the end of the cavity 123. This is the second configuration of the syringe.

As shown in FIG. 12D further movement of plunger 1212 causes the piston 129 to move within barrel 126 so that medicament is expelled from needle 128. Prior to this the needle 128 may have been inserted through the user's skin to enable the injection.

As shown in FIG. 12E after the injection has been performed the plunger 1212 may be moved back in the opposite longitudinal direction within cavity 1210. Friction between plunger 129 and the syringe barrel 126, or other conventional engagement, cause the entire syringe to be drawn back in the same direction along cavity 123 until the needle 128 is again within cavity 123, and is consequently shielded by the sides of the cavity, protecting the user, or others, from accidental needle injury. At the extreme end of this movement the barrel 126 may abut against end stops 1220.

FIG. 12F shows a safety feature. The connection between plunger 1212 and piston 129 is made such that the strength of the connection is sufficient to cause the barrel 126 to be drawn back to the position as shown in FIG. 12D, but on application of further force to the plunger 1212 to urge it in the direction away from the syringe the connection is broken, so that the syringe cannot be re-used. To facilitate this the piston 129 may be provided with a means (not shown) e.g. conventional one-way barbs, to prevent the piston from being moved back within barrel 126. As a further or alternative safety feature the cavity 123 and barrel 126 may be provided with locking features (not shown) to lock the barrel 126 in the position shown in FIG. 12F.

To further ensure sterility the device 120 may be provided for use sealed in a sterile pack (not shown).

As a further safety feature the syringe may be biased e.g. by a spring (not shown) for example operative within cavity 123 which may act to return the barrel 126 from the second configuration as shown in FIGS. 12C and 12D toward its first configuration as shown in FIG. 12D. Alternatively such a spring (not shown) may be operative within cavity 1210 to act upon the plunger 1212.

FIG. 13 shows such a device 130 overall. As can be seen the construction of the device is analogous to that of the device 120, but the body is provided with two (one may be used) air cavities 131 extending longitudinally in the body 132, parallel to the syringe cavity 133. Connected to, e.g. integrally made with plunger 134 are two air pistons 135 which are each slideably moveable within a cavity 131, and which each conform substantially to the cross section of the cavity 131 in which it is moveable. As the plunger 134 moves in the injecting direction to operate the device 130 analogously to the device of FIG. 12 under an urging force applied by the user, the pistons 135 also move within their cavities 131, to compress air within the cavities 131. When this urging force is released the expansion of the compressed air in cavities 131 forces the pistons 135 back in the opposite direction to return the syringe to its first configuration analogous to FIG. 12E of FIG. 12F.

FIG. 14 shows a device 140 overall in which two devices 120 are linked together along their long sides by a frangible link 141. More than two such devices 120 may be linked in such a manner. This enables plural devices 120 each containing one unit of a multi-unit dose of the medicament to be provided to a user. The link 141 may be foldable, so that the multiple devices 120 may be provided in a folded flat arrangement, retaining the convenient credit card shaped flat format.

FIG. 15 shows a device 150 overall which incorporates two syringes 151, 152 analogous in construction and general operation to those (126,127,128,129) above and which may be used sequentially to administer sequential doses of the same or a different medicament. The body 153 of device 150 incorporates a cavity 154 within which is located a plunger button 155 (shown of circular form but could be other shape) which is mounted on longitudinal axle 156. Axle 156 allows the button 155 both to be longitudinally along the axle 156, i.e. as a rail, in the injecting direction shown by the arrow and reciprocally in the return direction. Axle 156 also allows the button 155 to rotate about the axis of the axle 156, i.e. out of the plane of the drawing, through at least 180°, cavity 154 being open to allow this, for example having a suitable orifice in any cavity wall, or a peel-off cover. Connected to, e.g. integrally made with, button 155 is a plunger 157, and a spring mount 159 on which is mounted a spring 159 (shown as a coil spring, other types of bias means could be used).

In use the button 155 is moved by the user in the direction of the arrow. This causes plunger 157 to move toward piston 1510 of syringe 151, to engage with piston 1510, and to cause the syringe to operate in a manner analogously to that above. Simultaneously this movement of button 155 causes spring 59 to be compressed between mount 158 and the end surface of cavity 154. When urging pressure is released from button 155 the spring 159 expands to urge button 155 back in the opposite direction to the arrow, pulling syringe 151 back at the same time, analogously as above. As above the engagement between plunger 157 and piston 1510 is such that as the syringe barrel reaches its extreme backward position the plunger 157 is disengaged from piston 1510, for example there may be a weak snap-fit or ball-and-socket engagement between plunger 157 and piston 1510.

With plunger 157 disengaged from piston 1510 the button 155 may then be rotated on axle 156 so that the positions of plunger 157 and spring 159 are exchanged. The button 155 may then be moved again in the direction of the arrow so that plunger 157 engages with piston 1511 of syringe 152 to operate syringe 152 in a manner analogous to syringe 151.

1-23. (canceled)

24. An injector device comprising:
a substantially planar body having a syringe cavity and incorporating a syringe, the syringe comprising:
a barrel defining a reservoir for an injectable medicament;
an injection needle in communication with the reservoir such that medicament content in the reservoir can be expelled from the reservoir via the needle;
and an internal piston which can be moved in an injecting direction along inside the barrel toward the needle to inject the medicament content through the needle, the needle having a point and the syringe being slideably moveable within the syringe cavity in the body between a first configuration in which the body and the syringe are substantially coplanar and the needle has at least its point shielded, and a second configuration in which the needle is exposed for use for an injection to be administered;
wherein the syringe cavity is in communication with a plunger cavity, coaxial with the syringe cavity and contains a plunger shaft connected to the internal piston, the plunger shaft being moveable within the cavity by an urging force applied by a user thereto toward the syringe to urge the internal piston in the injecting direction;
a mechanism such that the plunger shaft is able to move the internal piston within the barrel in the injecting direction toward the needle, and when the plunger shaft is subsequently moved in the opposite direction from the injecting direction the plunger shaft and/or the internal piston engage with the barrel to move the syringe back towards its first configuration; and
wherein the connection between the plunger shaft and the internal piston is severable after use to inhibit re-use.

25. An injector device according to claim 24, wherein the syringe is biased to be returned toward its first configuration after the urging force urging the syringe toward its second configuration has been removed.

26. An injector device according to claim 25, wherein the syringe is biased by a spring.

27. An injector device according to claim 24, wherein in its first configuration at least the point of the needle is shielded by providing a sterile envelope around at least the point of the needle.

28. An injector device comprising two or more devices according to claim 24, linked together by a severable link.
